(19) United States
(12) Patent Application Publication
(10) Pub. No.: US 2017/0105923 A1
(43) Pub. Date: Apr. 20, 2017

(54) Silicone Film Formers In Antiperspirant And Deodorant Formulations

(71) Applicant: Dow Corning Corporation, Midland, MI (US)

(72) Inventors: Lylenette Canfield, Midland, MI (US); Paige Krzyskowski, Freeland, MI (US)

(21) Appl. No.: 15/293,868

(22) Filed: Oct. 14, 2016

Related U.S. Application Data
(60) Provisional application No. 62/242,323, filed on Oct. 16, 2015.

(51) Int. Cl. A61K 8/894; A61K 8/891; A61K 8/58; A61Q 15/00; A61K 8/26

(52) U.S. Cl. CPC A61K 8/894; A61Q 15/00; A61K 8/26; A61K 8/585; A61K 8/891; A61K 2800/594

(57) ABSTRACT

Silicone film formers used in APDO formulations can reduce the transfer of silicone and active ingredients, thereby offering long wear to APDO, and transfer resistance of APDO from the skin while maintaining breathability of the skin.

### CROSS-REFERENCE TO RELATED APPLICATIONS

This application claims the benefit of U.S. Provisional Appl. Ser. No. 62/242,323, filed Oct. 16, 2015, which is incorporated by reference as if fully set forth herein.

### BACKGROUND

Silicone film formers are used in many personal care products. For example, silicone film formers are known to promote durability and long wear in color cosmetics and wash off resistance in sun care products. There are several properties of films that are relevant to such products including water and sebum repellency, permeability to water vapor, film flexibility, film integrity and film durability.

Permeability to water vapor is a particularly important attribute for color cosmetics and skin care products. For example, permeability to water vapor impacts comfort of wear on the skin and allows breathability of the skin. For antiperspirants and deodorants (APDO), this property relates to the efficacy of the material.

### BRIEF SUMMARY OF THE INVENTION

The formulation of the invention includes a silicone film former and one or more ingredients wherein the silicone film former improves resistance to the transference of silicone in the film former as well as the one or more ingredients away from a surface of a substrate to which the formulation is applied. The silicone film former is a resin, a wax or a combination thereof. The ingredients are active ingredients, inactive ingredients or a combination thereof.

### BRIEF DESCRIPTION OF THE DRAWINGS

FIG. 1 illustrates results from water permeability tests on common silicones that act as film formers.

FIG. 2. illustrates transfer resistance of silicon from a silicone film coated on a skin-mimicking substrate.

FIG. 3. illustrates the silicon concentration before and after 10 abrasions.

FIG. 4. illustrates the aluminum intensity (kcps) before and after 10 abrasion.

### DETAILED DESCRIPTION OF THE INVENTION

The following is illustrative of the use of silicone film formers in antiperspirant and deodorant (APDO) formulations. As shown and described herein, these film formers improve resistance to the transference of silicon and other actives, e.g. aluminum, from one surface to another.

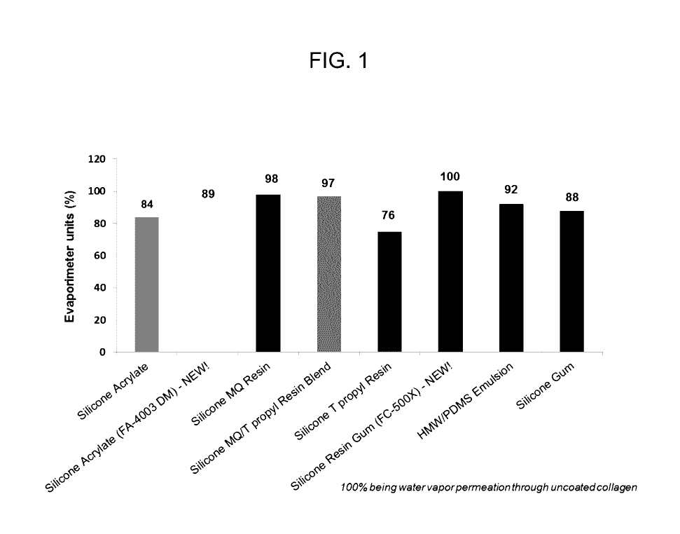

FIG. 1 illustrates the permeability of films coated on collagen. More specifically, the film formers are all 20 weight percent in a solvent carrier coated on collagen (50 nm). The silicone acrylate is Dow Corning® FA-4001 CM (INCI name Cyclopentasiloxane (and) Acrylates/Polytrimethylsiloxymethacrylate Copolymer) or FA-4002 ID (INCI name Isododecane (and) Acrylates/ Polytrimethylsiloxymethacrylate Copolymer). The silicone acrylate “new” is Dow Corning® FA-4003 DM (INCI name Dimethicone (and) Acrylates/Polytrimethylsiloxymethacrylate Copolymer). The silicone MQ resin is Dow Corning® MQ-1600 (INCI name Trimethylsiloxysilicate) or any variation such as Dow Corning® RSN-0749 (INCI name Cyclopentasiloxane (and) Trimethylsiloxysilicate) or 593 fluid (INCI name Dimethicone (and) Trimethylsiloxysilicate). The silicone MQ/T propyl resin blend is Dow Corning® MQ-1640 (INCI name Trimethylsiloxysilicate (and) Polypropylsilsesquioxane). The silicone T propyl resin is Dow Corning® 670 (INCI name Cyclopentasiloxane (and) Polypropylsilsesquioxane) or 680 ID fluid (INCI name Polypropylsilsesquioxane (and) Isododecane). The silicone resin gum is Dow Corning® FC-5002 ID (INCI name Isododecane (and) Trimethylsiloxysilicate/Dimethiconol Crosspolymer) or other variations of Dow Corning resin gum in different carrier fluid.

Permeability was tested using a Payne cup method, based on ASTM standard E96/E 96M-05 evaluating the water vapor permeability of polymer films, adapted to personal care and topical applications. Diluted products were coated on Naturin collagen (Naturin Viscofan GmbH, Germany) using an automatic coating system (K Control Coater Model 101, RK PrintCoat Instruments Ltd., United Kingdom) and a 50 μm gap quadruple applicator device (TQC B.V., The Netherlands). Dry films were then mounted on Payne cups (Elcometer, Belgium) partially filled with water (ca. 3 g). This system was subsequently placed under controlled temperature (31° C.) and humidity conditions to monitor the rate of water evaporation through the coated film (membrane) system

The higher percentage value indicates greater permeability of the film to water vapor, with 100% being full permeation of water vapor through untreated collagen. Although these resinous materials are all know in the industry to be highly permeable to water vapor, the data indicates that the presence of Tpropyl moieties on a silicone resin does impact the resin's permeability properties.

Silicone film formers, e.g. in the form of resins and waxes, are well known in the art for reducing transfer in color cosmetics and imparting wash off resistance in sun care products. These silicone film formers also provide similar attributes in antiperspirants and deodorants. More specifically, they keep active and silicone ingredients on the skin.

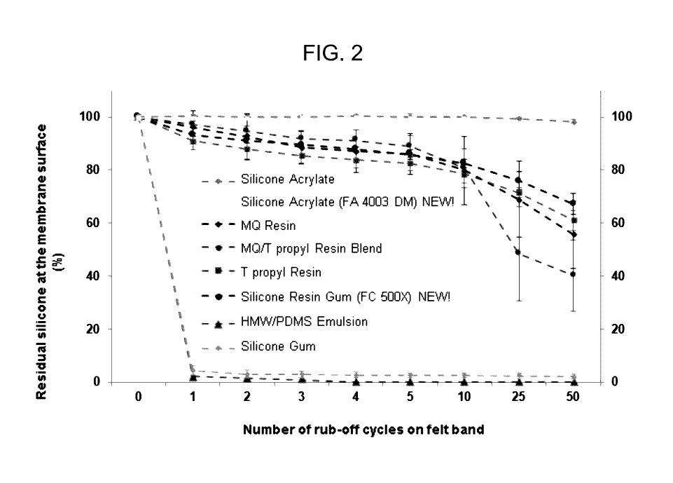

FIG. 2 illustrates rub-off, i.e. transfer resistance, of a film coated on a skin-mimicking substrate. Resistance is measured by x-ray fluorescence (XRF) after friction cycles using a washability tester/felt band, and specifically a Braive Washability instrument. FIG. 2 illustrates results for transfer resistance of neat materials (not in formulation). Film formers were diluted at 20 weight percent active level in a solvent carrier and coated on collagen (50 μm).

The silicone acrylate is Dow Corning® FA-4001 CM (INCI name Cyclopentasiloxane (and) Acrylates/Polytrimethylsiloxymethacrylate Copolymer) or FA-4002 ID (INCI name Isododecane (and) Acrylates/Polytrimethylsiloxymethacrylate Copolymer). The silicone acrylate “new” is Dow Corning® FA-4003 DM (INCI name Dimethicone (and) Acrylates/Polytrimethylsiloxymethacrylate Copolymer). The silicone MQ resin is Dow Corning® MQ-1600 (INCI name Trimethylsiloxysilicate) or any variation such as Dow Corning® RSN-0749 (INCI name Cyclopentasiloxane (and) Trimethylsiloxysilicate) or 593 fluid (INCI name Dimethicone (and) Trimethylsiloxysilicate). The silicone MQ/T propyl resin blend is Dow Corning® MQ-1640 (INCI name Trimethylsiloxysilicate (and) Polypropylsilsesquioxane). The silicone T propyl resin is Dow Corning® 670 (INCI name Cyclopentasiloxane (and) Polypropylsilsesquioxane) or 680 ID fluid (INCI name Polypropylsilsesquioxane (and) Isododecane). The silicone resin gum is Dow Corning® FC-5002 ID (INCI name Isododecane (and) Trimethylsiloxysilicate/Dimethiconol Crosspolymer) or other variations of Dow Corning resin gum in different carrier fluid.

The diluted products were coated on to Naturin collagen using an automatic coating system and a 50 μm gap quadruple applicator. The resistance to rub-off was evaluated after exposing the dry films to a maximum of 50 rub-off cycles on a felt band (Ideal Felt N.V., Belgium) using a washability tester (Braive Instruments S.A., Belgium). Quantification of residual Si at the collagen surface was performed using an X-ray fluorescence analyzer (XRF) (Oxford Instruments plc, United Kingdom).

The results of the aforementioned test illustrate the amount of silicon that remains on the substrate after a number of abrasions and the higher percentage value indicates greater resistance to rub-off. More specifically, as shown, the silicone acrylates have the best durability or resistance to rub-off/transfer. Other top performers include MQ or MQ and T propyl containing resins and resin blends (such as Dow Corning MQ-1600, MQ-1640, 670, and 680). Whereas films made from high-molecular weight PDMS, irrespective of their physical form (e.g. neat versus emulsion) exhibited excessively poor adhesion properties to the skin mimicking substrate.

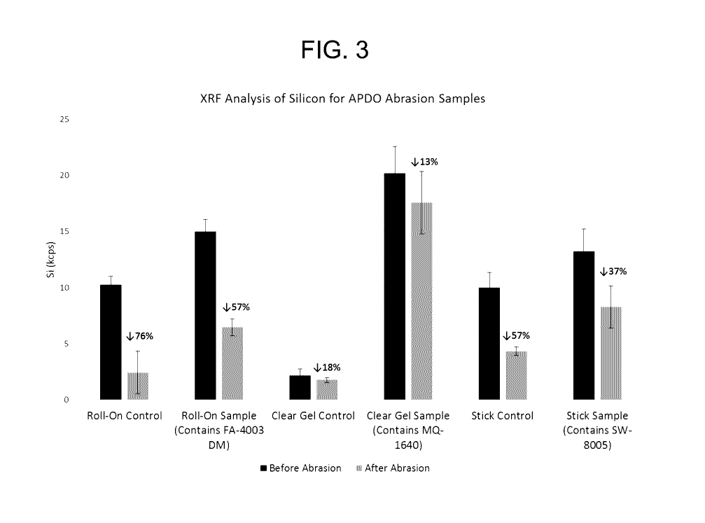

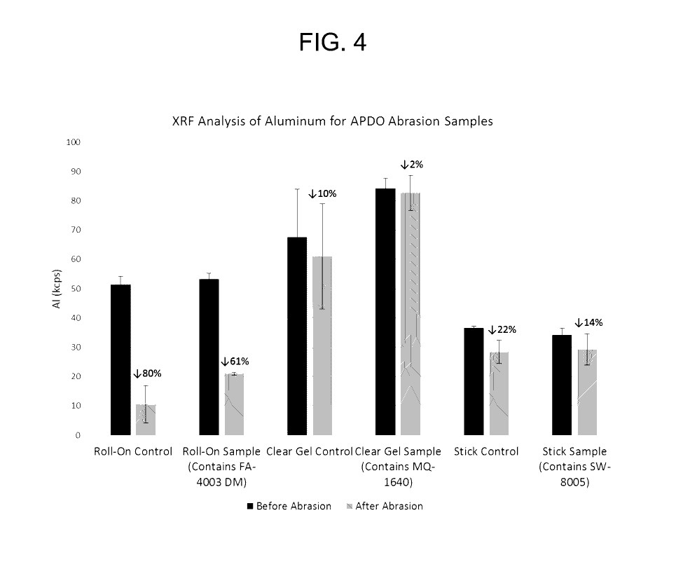

The following will demonstrate two different APDO formulations utilizing two different classes of silicone film forming materials where the impact on transfer resistance is evident. The silicone film durability tests (FIG. 1) were used to hypothesize these benefits in APDO. Example formulations and data for transfer resistance obtained using XRF can be found in Table 1-3 and FIG. 3-4.

Gel APDO Formulation

The formulation in Table 1 below demonstrates the ability of silicone resins to resist transfer when used in a water in oil, clear gel APDO. The use of Dow Corning® MQ-1640 is illustrated but substitutions include but are not limited to the following: Dow Corning® MQ-1600 (INCI: trimethylsiloxysilicate), Dow Corning® RSN-0749 (INCI: cyclopentasiloxane (&) trimethylsiloxysilicate), Dow Corning® 593 Fluid (INCI: dimethicone (&) trimethylsiloxysilicate), Dow Corning® 670 (cyclopentasiloxane (&) polypropylsilsesquioxane), or Dow Corning® 680 ID (INCI: isododecane (&) polypropylsilsesquioxane). Other water-in-oil emulsifiers, other APDO actives, and other solvents and carrier fluids can also be used.

TABLE 1

| | | | |
|---|---|---|---|
| | | Sample | Control |
| Ingredient | Supplier/Trade Name | Weight % | Weight % |

Phase A

| | | | |
|---|---|---|---|
| Cyclopentasiloxane (and) PEG/PPG- | Dow Corning ® 5225C | 6.5 | 6.5 |
| 18/18 Dimethicone | Formulation Aid | | |
| Cyclopentasiloxane | XIAMETER ® PMX- | 0 | 18.5 |
| | 245 | | |
| Trimethylsiloxysilicate (and) | Dow Corning ® MQ- | 18.5 | 0 |
| polypropylsilsesquioxane (27% | 1640 Flake Resin + | | |
| solution in cyclopentasiloxane) | XIAMETER PMX-245 | | |

Phase B

| | | | |
|---|---|---|---|
| Aluminum Sesquichlorohydrate | Reach 301 Solution/ | 50 | 50 |
| | Reheis, Inc. | | |
| Deionized Water | | 10 | 10 |
| Propylene Glycol | Propylene Glycol/The | 15 | 15 |
| | Dow Chemical | | |
| | Company | | |

It is noted that propylene glycol is used in the above formula to help match the refractive index of the two phases in order to obtain a clear emulsion. The process for creating the above water-in-oil clear gel emulsion is as follows: combine phase A ingredients and mix; in a separate vessel combine phase B ingredients and mix; match the refractive index of phase B to phase A if desired; add phase B to phase A very slowly while maintaining turbulent mixing; mix at high shear and high speed for two minutes after addition or run through a homogenizer.

Stick APDO Formulation

The formulation in Table 2 demonstrates the ability of silicone resin-wax combinations used in APDO to facilitate transfer resistance. Wax materials also have shown duel function to also help build the structure of the stick APDO and in some cases enhance the sensory attributes. Other silicone waxes often used in stick APDO formulations include but are not limited to Dow Coming® 2503 cosmetic wax (INCI: stearyl dimethicone (&) octadecene), Dow Corning® 580 wax (INCI: stearoxytrimethylsilane (&) stearyl alcohol), and Dow Coming® AMS-C30 Wax (INCI: C30-45 alkyl methicone (&) C30-40 olefin). Other silicone film formers such as silicone gums, silicone resins, and silicone acrylates can also be incorporated into APDO stick formulations. A variety of APDO actives, waxes, powders, and other solvents and carrier fluids can also be used in similar stick APDO chassis.

TABLE 2

| | | | |
|---|---|---|---|
| | | Sample | Control |
| Ingredient | Supplier/Trade Name | Weight % | Weight % |

Phase A

| | | | |
|---|---|---|---|
| Hydrogenated Castor Oil | BASF Care | 5 | 5 |
| | Chemicals/Cutina HR | | |
| Stearyl Alcohol | BASF Care | 16 | 21 |
| | Chemicals/Lanette 18 | | |
| Dimethicone (and) Trisiloxane | XIAMETER ® PMX- | 48 | 48 |
| | 1184 Silicone Fluid | | |
| C30-45 Alkyldimethylsilyl | Dow Corning ® SW- | 5 | 0 |
| Polypropylsilsesquioxane (50% | 8005 C30 Resin Wax + | | |
| solution in cyclopentasiloxane) | XIAMETER ® PMX- | | |
| | 245 | | |

Phase B

| | | | |
|---|---|---|---|
| Aluminum Zirconium | Summit Reheis, Inc./ | 25 | 25 |
| Tetrachlorohydrex GLY | Reach AZP-908 SUF | | |
| Talc | Presperse, Inc./Talc | 1 | 1 |
| | Micro-Ace P-4 | | |

After formulation it was noted that the stick without the Dow Corning® SW-8005 did not stick together as well or smooth on to the collagen substrate as well. The procedure for the above APDO stick is as follows: heat phase A ingredients to 80-90° C. until completely melted and mix; add phase B while mixing; stir until the mixture is homogeneous; cool to 60° C. while mixing; and fill containers while hot.

Roll-On APDO Formulation

The formulation in Table 3 demonstrates the ability of silicone acrylate combinations used in APDO to facilitate transfer resistance. Silicone film forming materials also have shown duel function, also enhancing the sensory attributes. Other silicone acrylates or resins in solvent can be used in roll-on APDO formulations. These include but are not limited to Dow Coming® MQ-1600 Solid resin (INCI name Trimethylsiloxysilicate), Dow Corning MQ-1640 Flake Resin (INCI name Trimethylsiloxysilicate (and) polypropylsilsesquioxane), Dow Corning RSN-0749 (INCI name Cyclopentasiloxane (and) Trimethylsiloxysilicate), Dow Corning® 593 Fluid (INCI name Dimethicone (and) Trimethylsiloxysilicate), Dow Corning® FA-4001 CM (INCI name Cyclopentasiloxane (and) Acrylates/Polytrimethylsiloxymethacrylate Copolymer), Dow Corning® FA-4002 ID (INCI name Isododecane (and) Acrylates/Polytrimethylsiloxymethacrylate Copolymer), Dow Corning® FC-5002 ID Resin Gum (INCI name Isododecane (and) Trimethylsiloxysilicate/Dimethiconol Crosspolymer), Dow Corning® 670 (INCI name Cyclopentasiloxane (and) Polypropylsilsesquioxane), or Dow Corning 680 ID fluid (INCI name Polypropylsilsesquioxane (and) Isododecane). Other water-in-oil emulsifiers, other APDO actives, and other solvents and carrier fluids can also be used.

TABLE 3

| | | | |
|---|---|---|---|
| | Supplier/Trade | Sample | Control |
| Ingredient | Name | Weight % | Weight % |

Phase A

| | | | |
|---|---|---|---|
| Cetyl Diglyceryl | Dow Corning ® ES- | 2 | 2 |
| Tris(Trimethylsiloxy)silylethyl | 5600 Silicone | | |
| Dimethicone | Glycerol Emulsifier | | |
| Dimethicone | XIAMETER ® PMX- | 9 | 14 |
| | 200 Fluid 2 CS | | |
| Phenyl Timethicone | Dow Corning ® 556 | 2 | 2 |
| | Fluid | | |
| Dimethicone (and) | Dow Corning ® FA- | 5 | 0 |
| Acrylates/Polytrimethylsiloxymethacrylate | 4003 DM Silicone | | |
| Copolymer | Acrylate | | |

Phase B

| | | | |
|---|---|---|---|
| Aluminum Zirconium Octachlorohydrex | Reach AZO | 45 | 45 |
| Glycine (and) water | 956G/Summit | | |
| | Reheis, Inc. | | |
| Glycerin | | 5.5 | 5.5 |
| Propanediol | | 12.5 | 12.5 |
| Deionized Water | | 18.5 | 15.5 |
| Phenoxyethanol (and) Ethylhexylglycerin | | 0.5 | 0.5 |

The procedure for the roll-on APDO water-in-oil formulation in table 3 is as follows: combine phase A ingredients and mix; in a separate vessel combine phase B ingredients and mix; match the refractive index of phase B to that of phase A if desired; add phase B to phase A very slowly while maintaining turbulent mixing; and mix at high shear and high speed for two minutes after addition or run through a homogenizer.

X-Ray Fluorescence (XRF)

XRF was used to measure the fluorescent from silicon and from aluminum emitted after high-energy X-rays hit the surface of the collagen substrate used for the APDO. Collagen was used as the substrate. 0.1 g of APDO was applied evenly over the whole surface area of the substrate. Samples were allowed to dry down over night for the clear gel, roll-on, and stick formulas, then a die-cut was used to extract a circular sample for the XRF. Samples were read before abrasion and after 10 cycles (20 passes) of abrasion with 150 g weight using a Braive Washability tester. Results are shown in FIG. 3 and FIG. 4.

FIG. 3 illustrates the silicon intensity (kilocounts per second, kcps) before and after 10 abrasions. Calibration curves have shown direct correlation of kcps to concentration. Silicone is present in both the control and sample formulations but would be higher in sample formulations as additional silicone film former is added. Each bar represents an average value for 3 samples. Error bars represent the standard deviation. Control denotes formulations without any silicone film former. Sample denotes formulations with silicone film former. Formulations can be seen in Table 1-3. The control stick APDO, the control clear gel APDO, and the control roll-on APDO formulation had significant reduction in measurable Si compared to the representative sample APDO formulations containing silicone film formers. The improvement in measurable silicone remaining on the substrate when using a silicone film former ranged from 5% for the clear gel formulation to 19% for both the roll on and stick formulations.

FIG. 4. illustrates the aluminum intensity (kcps) before and after 10 abrasion. Aluminum is present at equal amounts in both the control and sample formulations. Each bar represents an average value for 3 samples. Error bars represent the standard deviation. Control denotes formulations without any film former. Sample denotes formulations with silicone film former. Formulations can be seen in Table 1-3. The control stick APDO, the control clear gel APDO, and the control roll on APDO all had significant reduction in the amount of measurable Al remaining on the substrate after abrasion verses the representative sample APDO formulations containing film former. The improvement in the measurable Al remaining on the substrate when using a silicone film former ranged from 8% for the clear gel and stick formulations to 19% for the roll on formulation.

As a result of the above, it is clear that silicone film formers used in APDO formulations can reduce the transfer of silicone and active ingredients such as Aluminum based antiperspirants salts, thereby offering long wear to APDO and reduction of transfer of the APDO from the skin while maintaining breathability of the skin.

1. A formulation comprising:
a silicone film former; and
one or more ingredients;
wherein the silicone film former improves resistance to the transference of silicone in the film former as well as the one or more ingredients away from a surface of a substrate to which the formulation is applied.

2. The formulation of claim 1, wherein the silicone film former is a silicone acrylate resin, silicone resin, a silicone wax or a combination thereof.

3. The formulation of claim 1, wherein the one or more ingredients are active ingredients, inactive ingredients or a combination thereof.

4. The formulation of claim 3, wherein the one or more ingredients are active ingredients.

5. The formulation of claim 4, wherein the one or more active ingredients in an aluminum based antiperspirant salt.

6. The formulation of claim 1, wherein the substrate is skin.

7. The formulation of claim 6, whereby breathability of the skin is maintained.
